(19) United States
(12) Patent Application Publication
(10) Pub. No.: US 2022/0304658 A1
(43) Pub. Date: Sep. 29, 2022

(54) Cordless Ultrasonic Device

(71) Applicant: Judith Sene, Kissimmee, FL (US)

(72) Inventor: Judith Sene, Kissimmee, FL (US)

(21) Appl. No.: 17/214,685

(22) Filed: Mar. 26, 2021

(51) Int. Cl. A61B 8/00; A61B 8/14

(52) U.S. Cl. CPC A61B 8/4472; A61B 8/4488; A61B 8/461; A61B 8/56; A61B 8/14; A61B 2560/0214

(57) ABSTRACT

A cordless ultrasound device for a medical examination procedure, the cordless ultrasound device including a main body, including a head portion, and a handle portion disposed on at least a portion of the head portion to extend away from the head portion and facilitate gripping thereof, a transducer disposed on at least a portion of the head portion to emit at least one ultrasonic wave therefrom, and a power indicator comprising a battery and disposed on at least a portion of the handle portion to indicate a power level of the battery.

### BACKGROUND

### 1. Field

The present general inventive concept relates generally to ultrasound, and particularly, to a cordless ultrasound device.

### 2. Description of the Related Art

Human innovation has led to development of enhanced imagery using ultrasound for non-invasive cardiac examination, vascular examination, abdominal examination, transvaginal examination, and many other tests in order to assess for disease and/or other abnormal health conditions. Non-invasive procedures are highly beneficial to patients because it minimizes pain and/or risk of infection that arise during invasive surgery.

However, current medical ultrasound devices have restricted maneuverability and/or a significant risk of contamination due to the devices and cords falling to the floor. Also, the cords are often draped on patients during medical exams resulting in unnecessary anxiety.

Therefore, there is a need for a cordless ultrasound device to improve portability and maneuverability during a medical procedure.

### SUMMARY

The present general inventive concept provides a cordless ultrasound device.

Additional features and utilities of the present general inventive concept will be set forth in part in the description which follows and, in part, will be obvious from the description, or may be learned by practice of the general inventive concept.

The foregoing and/or other features and utilities of the present general inventive concept may be achieved by providing a cordless ultrasound device for a medical examination procedure, the cordless ultrasound device including a main body, including a head portion, and a handle portion disposed on at least a portion of the head portion to extend away from the head portion and facilitate gripping thereof, a transducer disposed on at least a portion of the head portion to emit at least one ultrasonic wave therefrom, and a power indicator comprising a battery and disposed on at least a portion of the handle portion to indicate a power level of the battery.

The head portion may be at least one of a convex ultrasound probe, a linear ultrasound probe, and a phased array ultrasound probe.

The transducer may electronically adjust a direction of the at least one ultrasonic wave.

The cordless ultrasound device may further include an oscillator disposed within at least a portion of the handle portion to generate and transmit at least one electronic signal oscillating at an ultrasonic frequency toward the transducer.

The cordless ultrasound device may further include a display unit disposed on at least a portion of the handle portion to display at least one visual rendering thereon in response to the transducer receiving an echo of the at least ultrasonic wave.

The display unit may project a holographic image of the at least one visual rendering above an outer surface of the display unit.

### BRIEF DESCRIPTION OF THE DRAWINGS

These and/or other features and utilities of the present generally inventive concept will become apparent and more readily appreciated from the following description of the embodiments, taken in conjunction with the accompanying drawings of which:

FIG. 1 illustrates a front isometric view of a cordless ultrasound device, according to an exemplary embodiment of the present general inventive concept;

FIG. 2 illustrates a front isometric view of a cordless ultrasound device, according to another exemplary embodiment of the present general inventive concept;

FIG. 3 illustrates a front isometric view of a cordless ultrasound device, according to another exemplary embodiment of the present general inventive concept; and

FIG. 4 illustrates an elevational top view of a charger, according to an exemplary embodiment of the present general inventive concept.

### DETAILED DESCRIPTION

Various example embodiments (a.k.a., exemplary embodiments) will now be described more fully with reference to the accompanying drawings in which some example embodiments are illustrated. In the figures, the thicknesses of lines, layers and/or regions may be exaggerated for clarity.

Accordingly, while example embodiments are capable of various modifications and alternative forms, embodiments thereof are shown by way of example in the figures and will herein be described in detail. It should be understood, however, that there is no intent to limit example embodiments to the particular forms disclosed, but on the contrary, example embodiments are to cover all modifications, equivalents, and alternatives falling within the scope of the disclosure. Like numbers refer to like/similar elements throughout the detailed description.

It is understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art. However, should the present disclosure give a specific meaning to a term deviating from a meaning commonly understood by one of ordinary skill, this meaning is to be taken into account in the specific context this definition is given herein.

### LIST OF COMPONENTS

Cordless Ultrasound Device 100

Main Body 110

Head Portion 111

Handle Portion 112

Transducer 120

Oscillator 130

Protruding Surfaces 140

Display Unit 150

Power Button 160

Power Indicator 170

Cordless Ultrasound Device 200

Main Body 210

Head Portion 211

Handle Portion 212

Transducer 220

Oscillator 230

Protruding Surfaces 240

Display Unit 250

Power Button 260

Power Indicator 270

Cordless Ultrasound Device 200

Main Body 310

Head Portion 311

Handle Portion 312

Transducer 320

Oscillator 330

Protruding Surfaces 340

Display Unit 350

Power Button 360

Power Indicator 370

Charging Unit 400

Body 410

Charging Connector 420

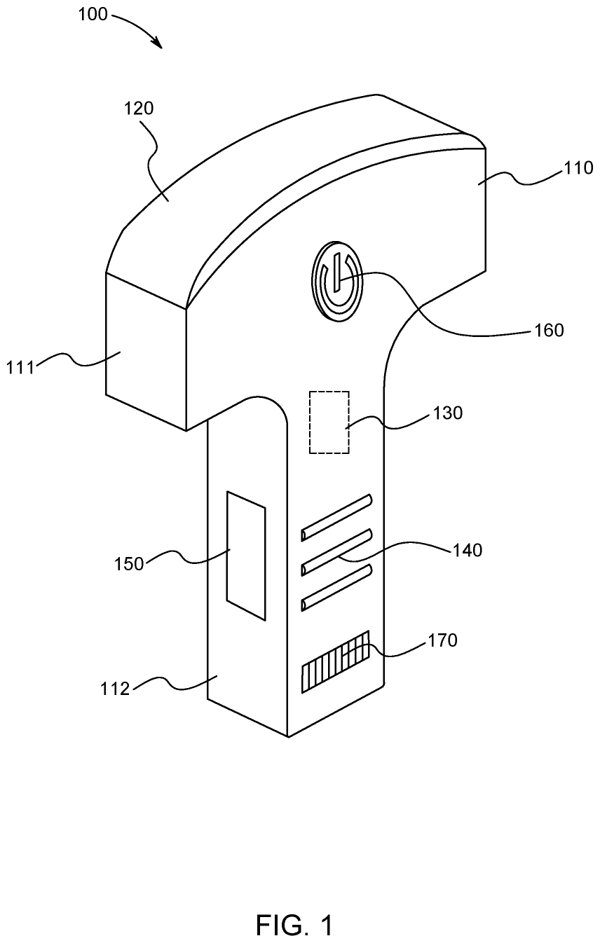

FIG. 1 illustrates a front isometric view of a cordless ultrasound device 100, according to an exemplary embodiment of the present general inventive concept.

The cordless ultrasound device 100 may be constructed from at least one of metal, plastic, wood, ceramic, glass, and rubber, etc., but is not limited thereto.

The cordless ultrasound device 100 may include a main body 110, a transducer 120, an oscillator 130, a plurality of protruding surfaces 140, a display unit 150, a power button 160, and a power indicator 170, but is not limited thereto.

The main body 110 may include a head portion 111 and a handle portion 112, but is not limited thereto.

Referring to FIG. 1, the head portion 111 is illustrated to be a convex ultrasound probe. However, the head portion 111 may be concave, rectangular, circular, conical, triangular, pentagonal, hexagonal, heptagonal, octagonal, or any other shape known to one of ordinary skill in the art, but is not limited thereto.

The handle portion 112 may be disposed on at least a portion of the head portion 111. Moreover, the handle portion 112 may extend away from the head portion 111 with respect to a direction, such that the handle portion 112 is elongated to facilitate gripping thereof. Also, the main body 110 may be covered with cover, such as a plastic and/or rubber cover with a lanyard.

The transducer 120 may be disposed on at least a portion of a first end (i.e. a front surface) of the main body 110. The transducer 120 may emit at least one ultrasonic wave therefrom. For example, the transducer 120 may emit the at least one ultrasonic wave toward a tissue of a body of a patient, such as a part of skin, a heart, an abdomen, and/or a vagina of the patient.

The oscillator 130 may include a beamforming oscillator, but is not limited thereto.

The oscillator 130 may be disposed within at least a portion of the handle portion 112. The oscillator 130 may generate and/or transmit at least one electronic signal comprising at least one pulse of a sine wave oscillating at an ultrasonic frequency toward the transducer, such that the transducer 120 may convert the at least one electronic signal into the at least one ultrasonic wave. Moreover, the transducer 120 may convert an alternating current (AC) received from the oscillator 130 into ultrasound.

Each of the plurality of protruding surfaces 140 may include a ridged surface. Additionally, the plurality of protruding surfaces 140 may be disposed on at least a portion of the handle portion 112 and extend away from an outer surface thereof. As such, the plurality of protruding surfaces 140 may increase friction against the handle portion 112. For example, the plurality of protruding surfaces 140 may prevent a finger of a user from moving away from the handle portion 112 due to increased friction.

The display unit 150 may be constructed as a plasma screen, an LCD screen, a light emitting diode (LED) screen, an organic LED (OLED) screen, a computer monitor, a hologram output unit, a sound outputting unit, an LED light, or any other type of device that visually or aurally displays data.

Referring again to FIG. 1, the display unit 150 is illustrated to be disposed on at least a portion of the handle portion 112. However, the display unit 150 may be disposed on any portion of the main body 110. The display unit 150 may display at least one visual rendering (e.g., an image, a picture, a video) thereon in response to the transducer 120 receiving an echo of the at least ultrasonic wave reflected back toward the transducer 120, such as from the tissue of the body of the patient.

Alternatively, the display unit 150 may project a holographic image of the at least one visual rendering above an outer surface of the display unit 150. As such, the display unit 150 may provide a three dimensional (3-D) render of the at least one visual rendering.

The power button 160 may be disposed on at least a portion of the head portion 111. The power button 160 may turn on the transducer 120, the oscillator 130, and/or the display unit 150 in response to depressing the power button a first time. Subsequently, the power button 160 may turn off the transducer 120, the oscillator 130, and/or the display unit 150 in response to depressing the power button a second time.

The power indicator 170 may include an illuminated light and a power source (e.g., a battery and/or a solar cell), but is not limited thereto. Also, the power indicator 170 may include a charging port disposed on a second end (i.e. a rear surface) of the main body 110.

The power indicator 170 may illuminate a first color (i.e. green) in response to determining the power source is at least a fifty percent capacity and/or is charging, such as in response to receiving an external energy source (e.g., sunlight) on the solar cell. However, the power indicator 170 may illuminate a second color (i.e. red) in response to determining the power source is less than fifty percent capacity and/or is not charging. As such, the power indicator 170 may indicate a power level of the battery.

Therefore, the cordless ultrasound device 100 may provide a portable and maneuverable means of performing an ultrasound procedure. Additionally, the cordless ultrasound device 100 may avoid contamination on surfaces because it does not depend on a cord for use. As such, the cordless ultrasound device 100 may avoid being disposed on the patient during a medical procedure.

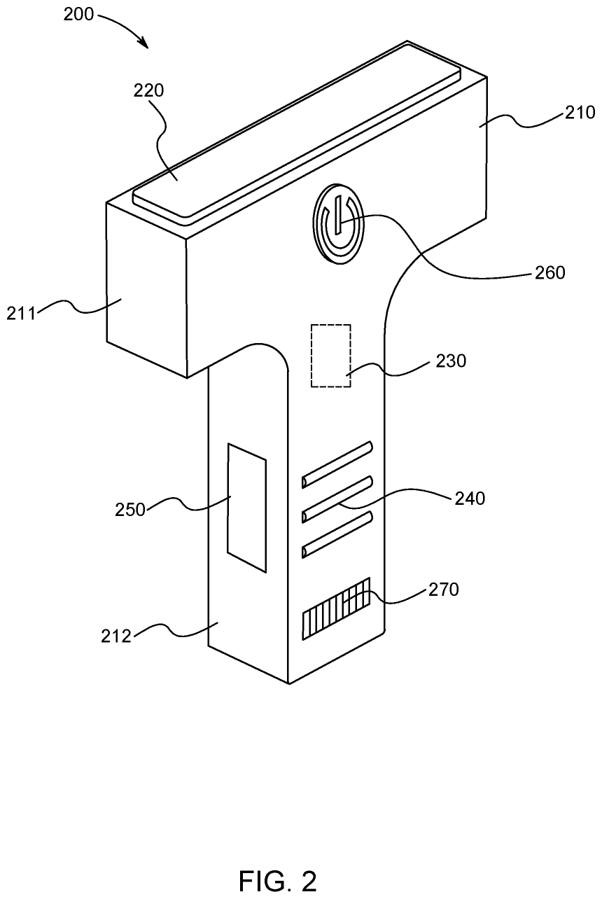

FIG. 2 illustrates a front isometric view of a cordless ultrasound device 200, according to another exemplary embodiment of the present general inventive concept.

The cordless ultrasound device 200 may be constructed from at least one of metal, plastic, wood, ceramic, glass, and rubber, etc., but is not limited thereto.

The cordless ultrasound device 200 may include a main body 210, a transducer 220, an oscillator 230, a plurality of protruding surfaces 240, a display unit 250, a power button 260, and a power indicator 270, but is not limited thereto.

The main body 210 may include a head portion 211 and a handle portion 212, but is not limited thereto.

Referring to FIG. 2, the head portion 211 is illustrated to be a linear ultrasound probe. However, the head portion 211 may be curved, circular, semi-circular with respect to the front view, conical, triangular, pentagonal, hexagonal, heptagonal, octagonal, or any other shape known to one of ordinary skill in the art, but is not limited thereto.

The handle portion 212 may be disposed on at least a portion of the head portion 211. Moreover, the handle portion 212 may extend away from the head portion 211 with respect to a direction, such that the handle portion 212 is elongated to facilitate gripping thereof. Also, the main body 210 may be covered with cover, such as a plastic and/or rubber cover with a lanyard.

The transducer 220 may be disposed on at least a portion of a first end (i.e. a front surface) of the main body 210. The transducer 220 may emit at least one ultrasonic wave therefrom. For example, the transducer 220 may emit the at least one ultrasonic wave toward a tissue of a body of a patient, such as a part of skin, a heart, an abdomen, and/or a vagina of the patient.

The oscillator 230 may include a beamforming oscillator, but is not limited thereto.

The oscillator 230 may be disposed within at least a portion of the handle portion 212. The oscillator 230 may generate and/or transmit at least one electronic signal comprising at least one pulse of a sine wave oscillating at an ultrasonic frequency toward the transducer, such that the transducer 220 may convert the at least one electronic signal into the at least one ultrasonic wave. Moreover, the transducer 220 may convert an alternating current (AC) received from the oscillator 230 into ultrasound.

Each of the plurality of protruding surfaces 240 may include a ridged surface. Additionally, the plurality of protruding surfaces 240 may be disposed on at least a portion of the handle portion 212 and extend away from an outer surface thereof. As such, the plurality of protruding surfaces 240 may increase friction against the handle portion 212. For example, the plurality of protruding surfaces 240 may prevent a finger of a user from moving away from the handle portion 212 due to increased friction.

The display unit 250 may be constructed as a plasma screen, an LCD screen, a light emitting diode (LED) screen, an organic LED (OLED) screen, a computer monitor, a hologram output unit, a sound outputting unit, an LED light, or any other type of device that visually or aurally displays data.

Referring again to FIG. 2, the display unit 250 is illustrated to be disposed on at least a portion of the handle portion 212. However, the display unit 250 may be disposed on any portion of the main body 210. The display unit 250 may display at least one visual rendering (e.g., an image, a picture, a video) thereon in response to the transducer 220 receiving an echo of the at least ultrasonic wave reflected back toward the transducer 220, such as from the tissue of the body of the patient.

Alternatively, the display unit 250 may project a holographic image of the at least one visual rendering above an outer surface of the display unit 250. As such, the display unit 250 may provide a three dimensional (3-D) render of the at least one visual rendering.

The power button 260 may be disposed on at least a portion of the head portion 211. The power button 260 may turn on the transducer 220, the oscillator 230, and/or the display unit 250 in response to depressing the power button a first time. Subsequently, the power button 260 may turn off the transducer 220, the oscillator 230, and/or the display unit 250 in response to depressing the power button a second time.

The power indicator 270 may include an illuminated light and a power source (e.g., a battery and/or a solar cell), but is not limited thereto. Also, the power indicator 270 may include a charging port disposed on a second end (i.e. a rear surface) of the main body 210.

The power indicator 270 may illuminate a first color (i.e. green) in response to determining the power source is at least a fifty percent capacity and/or is charging, such as in response to receiving an external energy source (e.g., sunlight) on the solar cell. However, the power indicator 270 may illuminate a second color (i.e. red) in response to determining the power source is less than fifty percent capacity and/or is not charging. As such, the power indicator 270 may indicate a power level of the battery.

Therefore, the cordless ultrasound device 200 may provide a portable and maneuverable means of performing an ultrasound procedure. Additionally, the cordless ultrasound device 200 may avoid contamination on surfaces because it does not depend on a cord for use. As such, the cordless ultrasound device 200 may avoid being disposed on the patient during a medical procedure.

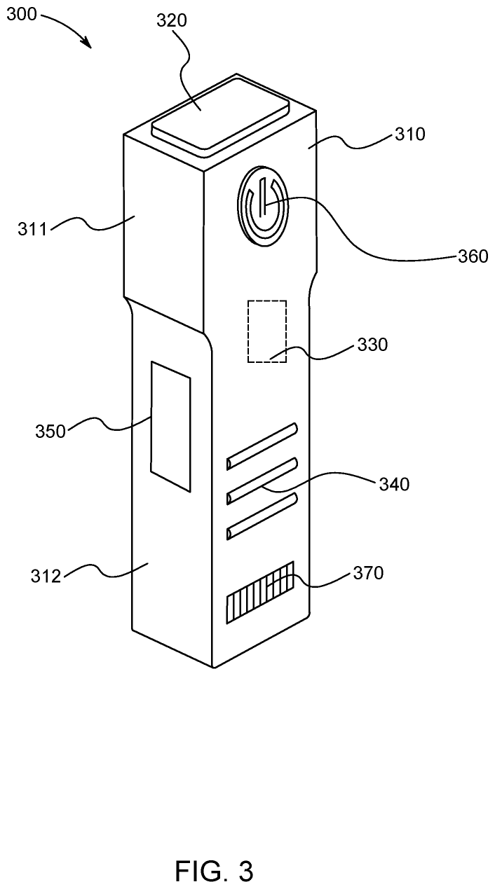

FIG. 3 illustrates a front isometric view of a cordless ultrasound device 300, according to another exemplary embodiment of the present general inventive concept.

The cordless ultrasound device 300 may be constructed from at least one of metal, plastic, wood, ceramic, glass, and rubber, etc., but is not limited thereto.

The cordless ultrasound device 300 may include a main body 310, a transducer 320, an oscillator 330, a plurality of protruding surfaces 340, a display unit 350, a power button 360, and a power indicator 370, but is not limited thereto.

The main body 310 may include a head portion 311 and a handle portion 312, but is not limited thereto.

Referring to FIG. 3, the head portion 311 is illustrated to be a phased array ultrasound probe.

The handle portion 312 may be disposed on at least a portion of the head portion 311. Moreover, the handle portion 312 may extend away from the head portion 311 with respect to a direction, such that the handle portion 312 is elongated to facilitate gripping thereof. Also, the main body 310 may be covered with cover, such as a plastic and/or rubber cover with a lanyard.

The transducer 320 may be disposed on at least a portion of a first end (i.e. a front surface) of the main body 310. The transducer 320 may emit at least one ultrasonic wave therefrom. For example, the transducer 320 may emit the at least one ultrasonic wave toward a tissue of a body of a patient, such as a part of skin, a heart, an abdomen, and/or a vagina of the patient.

Alternatively, the transducer 320 may be a plurality of transducers 320. As such, the plurality of transducers 320 may emit the at least ultrasonic wave and/or electronically adjust a direction of the at least one ultrasonic wave. It is important to note that his feature is not possible with the transducer 120 and/or the transducer 220, which only emit the at least one ultrasonic wave in a perpendicular direction away from the transducer, such that the at least one ultrasonic wave may move in the direction from the handle portion 112 toward the head portion 111. In other words, an orientation of the main body 110 and/or an orientation of the main body 210 may determine the direction the at least one ultrasonic wave moves. Similarly, the transducer 320 may emit the at least one ultrasonic wave in the direction from the handle portion 312 toward the head portion 311. Furthermore, each of the plurality of transducers 320 may be individually pulsed, such that each of the plurality of transducers may emit the at least one ultrasonic wave independently with respect to each other or simultaneously with respect to each other.

The oscillator 330 may include a beamforming oscillator, but is not limited thereto.

The oscillator 330 may be disposed within at least a portion of the handle portion 312. The oscillator 330 may generate and/or transmit at least one electronic signal comprising at least one pulse of a sine wave oscillating at an ultrasonic frequency toward the transducer, such that the transducer 320 may convert the at least one electronic signal into the at least one ultrasonic wave. Moreover, the transducer 320 may convert an alternating current (AC) received from the oscillator 330 into ultrasound.

Each of the plurality of protruding surfaces 340 may include a ridged surface. Additionally, the plurality of protruding surfaces 340 may be disposed on at least a portion of the handle portion 312 and extend away from an outer surface thereof. As such, the plurality of protruding surfaces 340 may increase friction against the handle portion 312. For example, the plurality of protruding surfaces 340 may prevent a finger of a user from moving away from the handle portion 312 due to increased friction.

The display unit 350 may be constructed as a plasma screen, an LCD screen, a light emitting diode (LED) screen, an organic LED (OLED) screen, a computer monitor, a hologram output unit, a sound outputting unit, an LED light, or any other type of device that visually or aurally displays data.

Referring again to FIG. 3, the display unit 350 is illustrated to be disposed on at least a portion of the handle portion 312. However, the display unit 350 may be disposed on any portion of the main body 310. The display unit 350 may display at least one visual rendering (e.g., an image, a picture, a video) thereon in response to the transducer 320 receiving an echo of the at least ultrasonic wave reflected back toward the transducer 320, such as from the tissue of the body of the patient.

Alternatively, the display unit 350 may project a holographic image of the at least one visual rendering above an outer surface of the display unit 350. As such, the display unit 350 may provide a three dimensional (3-D) render of the at least one visual rendering.

The power button 360 may be disposed on at least a portion of the head portion 311. The power button 360 may turn on the transducer 320, the oscillator 330, and/or the display unit 350 in response to depressing the power button a first time. Subsequently, the power button 360 may turn off the transducer 320, the oscillator 330, and/or the display unit 350 in response to depressing the power button a second time.

The power indicator 370 may include an illuminated light and a power source (e.g., a battery and/or a solar cell), but is not limited thereto. Also, the power indicator 370 may include a charging port disposed on a second end (i.e. a rear surface) of the main body 310.

The power indicator 370 may illuminate a first color (i.e. green) in response to determining the power source is at least a fifty percent capacity and/or is charging, such as in response to receiving an external energy source (e.g., sunlight) on the solar cell. However, the power indicator 370 may illuminate a second color (i.e. red) in response to determining the power source is less than fifty percent capacity and/or is not charging. As such, the power indicator 370 may indicate a power level of the battery.

Therefore, the cordless ultrasound device 300 may provide a portable and maneuverable means of performing an ultrasound procedure. Additionally, the cordless ultrasound device 300 may avoid contamination on surfaces because it does not depend on a cord for use. As such, the cordless ultrasound device 300 may avoid being disposed on the patient during a medical procedure.

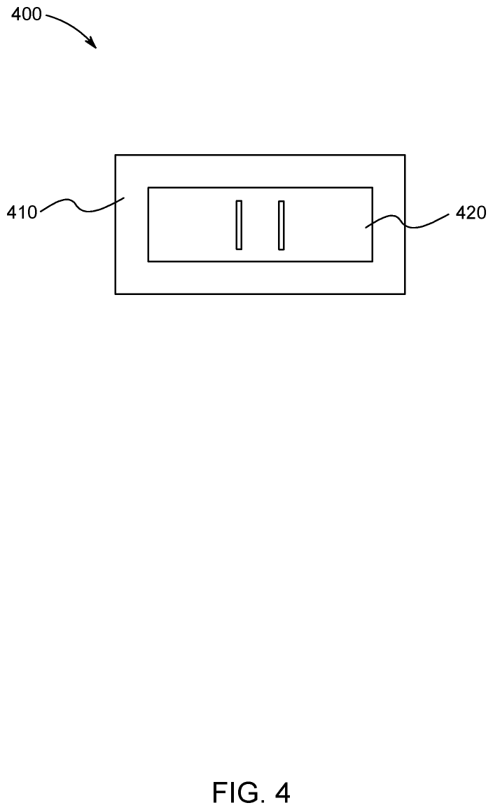

FIG. 4 illustrates an elevational top view of a charger 400, according to an exemplary embodiment of the present general inventive concept.

The charger 400 may include a body 410 and a charging connector 420, but is not limited thereto.

The body 410 may have any predetermined size and may include a power cord to connect to a power outlet.

The charging connector 420 may be disposed on at least a portion of the body 410. The charging connector 420 may receive the charging port of the power indicator 170, the charging port of the power indicator 270, and/or the charging port of the power indicator 370. Therefore, the charging connector 420 may send power to charge the battery of the power indicator 170, the battery of the power indicator 270, and/or the battery of the power indicator 370.

The present general inventive concept may include a cordless ultrasound device 100 for a medical examination procedure, the cordless ultrasound device 100 including a main body 110, including a head portion 111, and a handle portion 112 disposed on at least a portion of the head portion 111 to extend away from the head portion 111 and facilitate gripping thereof, a transducer 120 disposed on at least a portion of the head portion 111 to emit at least one ultrasonic wave therefrom, and a power indicator 170 comprising a battery and disposed on at least a portion of the handle portion 112 to indicate a power level of the battery.

The head portion 110 may be at least one of a convex ultrasound probe, a linear ultrasound probe, and a phased array ultrasound probe.

The transducer 120 may electronically adjust a direction of the at least one ultrasonic wave.

The cordless ultrasound device 100 may further include an oscillator 130 disposed within at least a portion of the handle portion 111 to generate and transmit at least one electronic signal oscillating at an ultrasonic frequency toward the transducer 120.

The cordless ultrasound device 100 may further include a display unit 150 disposed on at least a portion of the handle portion 112 to display at least one visual rendering thereon in response to the transducer 120 receiving an echo of the at least ultrasonic wave.

The display unit 150 may project a holographic image of the at least one visual rendering above an outer surface of the display unit 150.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

1. A cordless ultrasound device for a medical examination procedure, the cordless ultrasound device comprising:
a main body, comprising:
a head portion, and
a handle portion disposed on at least a portion of the head portion to extend away from the head portion and facilitate gripping thereof;
a transducer disposed on at least a portion of the head portion to emit at least one ultrasonic wave therefrom based on a sine wave oscillating at an ultrasonic frequency; and
a power indicator comprising a battery and disposed on at least a portion of the handle portion to indicate a power level of the battery.

2. The cordless ultrasound device of claim 1, wherein the head portion is at least one of a convex ultrasound probe, a linear ultrasound probe, and a phased array ultrasound probe.

3. The cordless ultrasound device of claim 1, wherein the transducer electronically adjusts a direction of the at least one ultrasonic wave.

4. The cordless ultrasound device of claim 1, further comprising:
an oscillator disposed within at least a portion of the handle portion to generate and transmit at least one electronic signal oscillating at an ultrasonic frequency toward the transducer.

5. The cordless ultrasound device of claim 1, further comprising:
a display unit disposed on at least a portion of the handle portion to display at least one visual rendering thereon in response to the transducer receiving an echo of the at least ultrasonic wave.

6. The cordless ultrasound device of claim 5, wherein the display unit projects a holographic image of the at least one visual rendering above an outer surface of the display unit.

7. A cordless ultrasound device for a medical examination procedure, the cordless ultrasound device comprising:
a main body, comprising:
a head portion, and
a handle portion disposed on at least a portion of the head portion to extend away from the head portion and facilitate gripping thereof;
a transducer disposed on at least a portion of the head portion to emit at least one ultrasonic wave therefrom by converting an alternating current into the at least one ultrasonic wave;
a plurality of protruding surfaces disposed on at least a portion of the handle portion to increase friction on the handle portion; and
a power indicator comprising a battery and disposed on at least a portion of the handle portion to indicate a power level of the battery.
